(19) United States
(12) Patent Application Publication
(10) Pub. No.: US 2023/0055537 A1
(43) Pub. Date: Feb. 23, 2023

(54) MULTI-MODAL NANOPORE SENSORS FOR NUCLEIC ACID SEQUENCING

(71) Applicant: University of South Florida, Tampa, FL (US)

(72) Inventors: Cai Mike Wang, Tampa, FL (US); Sameer Varma, Tampa, FL (US)

(21) Appl. No.: 17/890,143

(22) Filed: Aug. 17, 2022

Related U.S. Application Data
(60) Provisional application No. 63/234,653, filed on Aug. 18, 2021.

(51) Int. Cl. G01N 33/487; C12Q 1/6869; G01N 27/447

(52) U.S. Cl. CPC G01N 33/48721; C12Q 1/6869; G01N 27/44791; B82Y 15/00

(57) ABSTRACT

The present disclosure features an apparatus for identifying molecules, the apparatus including chambers configured to receive a conducting media; a membrane including a pore separating the chambers, a first set of electrodes in the chambers, a first current detector measuring an electrical signal between the first set of electrodes, a second set of electrodes electrically contacting the membrane, a second current detector configured measuring an electrical signal between the second set of electrodes, a plasmonic feature adjacent to the pore of the membrane, a light source configured to emit light onto the plasmonic feature, a light collector to collect the light scattered from the plasmonic feature, and a computing device configured to identify at least one attribute of a molecule that passes through the pore of the membrane.

### CROSS-REFERENCE TO RELATED APPLICATIONS

This application claims the benefit of priority to U.S. Application No. 63/234,653, filed on Aug. 18, 2021, the contents of which are hereby incorporated by reference.

### GOVERNMENT LICENSE RIGHTS

This invention was made with government support under 1944638 awarded by the National Science Foundation. The Government has certain rights in the invention.

### FIELD OF THE DISCLOSURE

The disclosure relates to nucleic acid sequencing and more specifically, to nucleic acid sequencing using electric signals and plasmon resonance detection in solid-state membranes.

### BACKGROUND

Nanopore sequencing is an approach that can be used in the sequencing of biopolymers. Specifically, nanopore sequencing can be used in the sequencing of nucleic acids, such as DNA or RNA. Using nanopore sequencing, a single molecule of DNA or RNA can be sequenced without the need for PCR amplification or chemical labeling of the molecule. Nanopore sequencing may include monitoring changes to electrical and/or optical signals as nucleic acids are passed through a solid-state nanopore. The electrical and/or optical signals can be analyzed to identify specific DNA or RNA sequence.

### SUMMARY

This disclosure describes a multi-modal nanopore sequencing technique. In some implementations, the technique provides a multi-modal nanopore sequencing that uses trans-membrane and trans-channel field effect electrical signals (e.g., conductance, inductance, capacitance, eddy current, induced EMF, magnetism, or other suitable electrical modalities) and surface plasmon resonance. The technique described herein can improve a nanopore sequencing that uses a single-modality of measurement of trans-membrane electrical signal. Such a single-modal nanopore sequencing relies on insulating thin films, which limits spatial-temporary accuracy and throughput. The multi-modal nanopore sequencing described herein can provide accuracy and repeatability for sequencing fidelity for timely epidemiological contact tracing, mutation monitoring, and therapeutic and vaccine development. Further, the multi-modal nanopore sequencing described herein can provide cross-validation between the signals, which can improve accuracy and increase translocation/read rate or throughput.

Some implementations of the multi-modal nanopore sequencing described herein combine trans-membrane approaches with trans-channel electrical signals and surface plasmon resonance. The multi-modal nanopore sequencing can use a broad spectrum of atomically thin, two-dimensional (2D) nanomaterials, which can have a thickness of less than a nanometer. Such atomic-scale 2D crystals can be manufactured to approach the length-scales of individual DNA/RNA nucleotides, and thus used for nucleotide sensing and DNA/RNA sequencing. Some implementations of the multi-modal nanopore sequencing described herein implement complementary multi-modal nano-optoelectronic measurements of the DNA/RNA nanopore translocation events via both the trans-membrane and the trans-channel electrical signals, in sync with near-field plasmonic excitation and sensing. The multi-modal nanopore sequencing improves spatial-temporal resolution and cross-verification of the precise genetic sequences. For example, the multi-modal nanopore sequencing can provide improved understandings of DNA/RNA interactions with nano-optoelectronic interfaces and lead to significant advances in rapid testing and de novo sequencing of novel pathogens.

In general, in a first aspect, the present disclosure features an apparatus for identifying molecules, the apparatus including a membrane that defines first and second chambers, the membrane including a pore that permits a molecule to pass through and move between the first and second chambers; a first set of electrodes disposed in the first and second chambers respectively and configured to generate a first electric field in a conducting media between the first and second chambers; a first current detector configured to measure a first current between the first set of electrodes; a second set of electrodes electrically contacting the membrane and configured to generate a second electric field across the pore of the membrane; a second current detector configured to measure a second current between the second set of electrodes; a plasmonic feature disposed on the membrane adjacent to the pore; a light source configured to emit light onto the plasmonic feature; a light collector configured to collect the light scattered from the plasmonic feature; and at least one computing device configured to identify at least one attribute of the molecule that passes through the pore of the membrane based on at least one of the first current, the second current, and the scattered light.

Embodiments of the apparatus for identifying molecules can include one or more of the following features. For example, the membrane can be a two-dimensional membrane. The two dimensional membrane can be graphene or silicon-based material. The membrane can be include of two or more stacked membranes.

In some embodiments, the electrodes can be insulated with a dielectric material.

In some embodiments, the plasmonic feature can be deposited on a membrane lacking pores. The plasmonic feature can be used to create the pore. The diameter of the plasmonic feature can be between 1 nm and 100 nm. The plasmonic feature can be a metallic plasmonic feature. The plasmonic feature can be deposited by chemical vapor deposition.

The molecule that passes through the pore of the membrane can be a nucleic acid. The at least one attribute of the molecule can be the sequence. The attribute of the molecule can include at least one of a size, a charge, or a weight of the molecule.

The at least one computing device can be configured to identify the at least one attribute of the molecule based on surface plasmon resonance.

In a second aspect, the present disclosure features a method for detecting a molecule attribute, the method including providing an apparatus including a membrane including a pore that permits a molecule to pass; a first set of electrodes disposed at opposite sides of the membrane and configured to generate a first electric field in the conducting media in a direction through the pore of the membrane; a second set of electrodes electrically contacting the membrane and configured to generate a second electric field across the pore of the membrane; a plasmonic feature disposed on the membrane adjacent to the pore; a light source configured to emit light onto the plasmonic feature; measuring at least one of a first signal representative of a first electric current or voltage supplying between the first set of electrodes; a second signal representative of a second electric current or voltage supplying between the second set of electrodes; or a third signal representative of a light scattered from the plasmonic feature deposited near the pore; and determining a molecule attribute based on the at least one of the first signal, the second signal, or the third signal.

The method can further include determining a size of the molecule based on the transit event. The molecule can be a nucleic acid. The nucleic acid can be DNA or RNA.

The method can further include determining a sequence of the nucleic acid based on the transit event.

The method can further include measuring the reflected plasmon resonance signal using microscopy.

Determining the molecule attribute can include determining the molecule attribute based on one or two of the first signal, the second signal, and the third signal; and verifying the molecule attribute based on the remaining of the first signal, the second signal, and the third signal.

Other advantages will be apparent from the description, the drawings, and the claims.

### BRIEF DESCRIPTION OF THE DRAWINGS

FIG. 1 is a schematic diagram of an example multi-modal nanopore sequencing system.

FIG. 2 is a schematic diagram of an example transmembrane electrical signal device.

FIG. 3 is a schematic diagram of an example trans-channel electrical signal device.

FIG. 4 is a schematic diagram of a surface plasmon detection device.

FIG. 5A is a schematic diagram of an example membrane with a deposited plasmonic feature

FIG. 5B is a schematic diagram of an example membrane with a pore formed from plasmonic feature heating and deformation.

FIG. 6 is an oblique view of an exemplary membrane with a plasmonic feature adjacent to a pore with a transiting double-stranded biopolymer.

FIG. 7A is an oblique view of an exemplary trans-channel electrical signal system with two plasmonic features adjacent to a pore with a transiting double-stranded biopolymer.

FIG. 7B is a top view of the exemplary trans-channel electrical signal and plasmonic system of FIG. 7A.

FIG. 8 is an oblique view of an exemplary trans-channel electrical signal system with two plasmonic features adjacent to a pore with a transiting single-stranded biopolymer.

In the figures, like symbols indicate like elements.

### DETAILED DESCRIPTION

In general, this disclosure relates to multi-modal nanopore sequencing techniques. Some implementations of the techniques include multi-modal nanopore sequencing systems and modes that use a combination of a trans-membrane and trans-channel field effect electrical signals (e.g., conductance, inductance, capacitance, eddy current, induced EMF, magnetism, or other suitable electrical modalities), and an optical signal (e.g., surface plasmonic resonance, fluorescence spectroscopy, Raman spectroscopy, Rayleigh scattering spectroscopy, Fourier transform infrared spectroscopy, UV-visible spectroscopy, scattering-type, or aperture-type scanning near-field optical microscopy). The multi-modal nanopore sequencing techniques described herein can provide accuracy and repeatability for sequencing fidelity for timely epidemiological contact tracing, mutation monitoring, and therapeutic or vaccine development.

For example, the multi-modal nanopore sequencing system can use a membrane made of one or more atomically thin, two-dimensional (2D) nanomaterials. For example, the material(s) of the membrane can have a thickness of equal to or less than the size of a molecule being tested, such as a nucleotide of a biopolymer. Such atomic-scale 2D materials can be manufactured to approach the length-scales of individual DNA/RNA nucleotides, and thus used for nucleotide sensing and DNA/RNA sequencing. Some implementations of the multi-modal nanopore sequencing described herein implement complementary multi-modal nano-optoelectronic measurements of the DNA/RNA nanopore translocation events via both the trans-membrane and the trans-channel electrical signals, in sync with near-field plasmonic excitation and sensing. The multi-modal nanopore sequencing improves spatial-temporal resolution and cross-verification of the precise genetic sequences. For example, the multi-modal nanopore sequencing can provide improved understandings of DNA/RNA interactions with nano-optoelectronic interfaces and lead to significant advances in rapid testing and de novo sequencing of potential emerging target pathogens, such as SARS-CoV-2.

The multi-modal nanopore sequencing system described herein include one or more membranes (e.g., one or more, two or more, three or more, four or more, or five or more membranes). In general, the one or more membrane(s) may be electrically insulating or semiconducting and impermeable to ions and other components of the surrounding electrolyte solution. The membrane of the multi-modal nanopore detection system can be made of a material that is non-conductive (e.g., semi-conductive, or insulative). The membrane material should be chosen such that the membrane is generally impermeable to solution components, is electrically insulating such that the chambers are not electrically connected (e.g., “shorted”), and the electrically conductive path is through the nanopore. For example, the membrane 106 can be composed of a dielectric material such as silicon nitride (e.g., SiNx). In some embodiments, the membrane can be a two dimensional material (e.g., graphene). In some embodiments, the membrane can be made of a three dimensional material (e.g., crystalline, amorphous, polymeric (PEDOS, PEDOT), elastomeric). The membranes can be made of substantially the same, or substantially different materials.

Each membrane can include one or more pores (e.g., one or more, two or more, three or more, four or more, or five or more) that enable one or more molecule(s) to pass through. The pores of the membrane can be of a size such that molecules or ions are allowed to flow through the pores but may not allow bulk material transfer. In general, the lower limit of the diameter of the pore is defined by a single atomic vacancy in the membrane. In general, the pore size can be chosen based upon the target biopolymer such that the pore is large enough to allow a single molecule to pass through while disallowing multiple simultaneous molecular transits (e.g., known as “crowding”).

The pore size range within the one or more membrane can include, but is not limited to, 0.1 nm to 100 nm (e.g., 0.1 nm to 100 nm, 20 nm to 100 nm, 40 nm to 100 nm, 60 nm to 100 nm, 80 nm to 100 nm, 0.1 nm to 80 nm, 0.1 nm to 60 nm, 0.1 nm to 40 nm, or 0.1 nm to 20 nm). In embodiments where there are one or more pores in each of the membranes, the pores can be of the same or of different sizes. Various modes known to the field can be used to create pores within the membrane. Example modes to create the pores can include, but are not limited to, beam lithography (e.g., optical, electron, or ion), plasma etching, electrochemical etching, Moiré interference patterning, or epitaxial growth.

The multi-modal nanopore sequencing system further includes a transit detection system that implements one or more detection modes. For example, the transit detection system can perform multiple modes of detection for charged molecules that pass through the pore of the membrane. Such multiple modes of detection can be performed concurrently or in predetermined sequence. In some implementations, the system includes a multi-modal nanopore system that provides three different modes of detection. For example, the multi-modal nanopore detection system can include a transmembrane electrical signal (e.g., ionic current) mode, a trans-channel electrical signal (e.g., conductance) mode, and an optical plasmon resonance mode. The three modes of the multi-modal nanopore detection system can function together or independently to detect the transit of a molecule through the pore in the membrane.

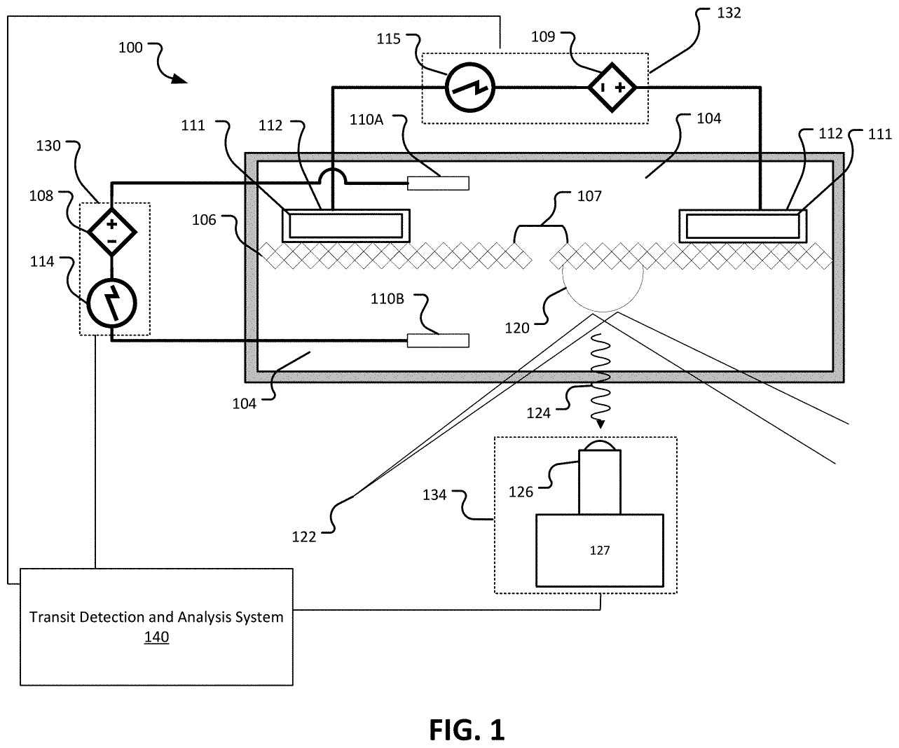

FIG. 1 depicts an exemplary multi-modal nanopore sequencing system 100 that can perform multiple modes to detect a molecule, such as a transmembrane electrical signal mode, a trans-channel electrical signal mode, and an optical plasmon resonance mode. In some implementations, the multi-modal nanopore sequencing system 100 can include chambers 104 separated and electrically insulated by a membrane 106. The volume of the chambers 104 can be filled with a solution that can contain ions (e.g., atoms or molecules that have lost or gained electrons) and can be electrically conductive (e.g., a conducting media). The solution can be a liquid or a gel (e.g., agar gel, agarose gel, polyacrylamide gel, or sephadex gel). The ions can be monoatomic, or polyatomic. The ions can be organic molecules or inorganic. In one example, the conducting media can contain sodium ions (e.g., saline solution, or phosphate buffered saline). The membrane 106 can include one or more pores 107 as described above.

The multi-modal nanopore sequencing system 100 can further include a first subsystem 130 (e.g., a transmembrane electrical signal system) for applying an electric potential to the conducting media. The first subsystem 130 can be configured to perform the transmembrane electrical signal mode, as described herein. In some implementations, the subsystem can include a first power supply 108 that is connected to one or more conductive electrodes 110 and configured to apply an electric potential between the conductive electrodes 110. For example, the electrodes 110 connected to the first power supply 108 can include a first electrode 110A and a second electrode 110B (collectively, 110). The first electrode 110A can be at least partially immersed into the conducting media in one compartment and the second electrode 110B can be at least partially immersed into the conducting media of the second compartment. The first subsystem 130 can further include an electrical measurement device 114 for the detection and measurement of the electrical signal flowing between the first and second electrodes 110. The combination of electrodes 110, power supply 108, and electrical measurement device 114 can constitute an electric circuit for the transmembrane electrical signal subsystem 130d. In general, the electrical measurement device 114 can be an ammeter or a potentiometer, thereby constituting a potentiostatic or galvanostatic electric circuit, respectively. FIG. 1 depicts the electrical measurement device 114 electrically connected in series with the power supply 108 but the device 114 can also be connected in parallel.

Referring still to FIG. 1, the multi-modal nanopore sequencing system 100 includes a second subsystem 132 (e.g., a trans-channel electrical signal system) for applying an electric potential to the membrane 106. The second subsystem 132 can be configured to perform the trans-channel electrical signal mode, as described herein. In some implementations, this can include a second power supply 109 that is connected to one or more conductive electrodes 111 and configured to apply an electric potential between the conductive electrodes 111. The conductive electrodes 111 can be arranged to electrically contact the membrane 106 so that a trans-channel electrical signal can be applied along the membrane 106 and detected by the configured electrical measurement device. In some implementations, the electrodes 111 are at least partially coated with an insulating material 112 to insulate the trans-channel electrical signal from the conducting media. Utilizing the second power supply 109 to generate an electric potential across the membrane 106 can complete an electric circuit for the trans-channel conductance mode subsystem 132.

In some implementations, the second subsystem 132 can include a second electrical measurement device 115 configured to measure the electrical signal of the membrane 106. The second electrical measurement device 115 can measure the electrical signal of the membrane 106 independent of the electrical signal measured by the first subsystem 130 (e.g., the transmembrane electrical signal system).

In the illustrated example, the first power source 108 and the second power source 109 are separately provided for the first subsystem 130 and the second subsystem 132, respectively. In other implementations, a single power source can function as the first power source 108 and the second power source 109 and supply power to the first subsystem 130 and the second subsystem 132. The first and second power sources 108 and 109 can be operated in a constant voltage mode, or a constant current mode.

Referring still to FIG. 1, the multi-modal nanopore sequencing system 100 further includes a third subsystem 134 (e.g., an optical plasmon resonance system). The third subsystem 134 can be configured to perform the optical plasmon resonance mode, as described herein. The third subsystem 134 can include one or more plasmonic feature 120 that is disposed near the pore 107 of the membrane 106. Embodiments featuring two or more plasmonic features 120 can be disposed near the pore 107 on the same, or on opposing sides of the membrane 106. Examples of plasmonic feature include a nanoparticle, patch, or spot. The plasmonic feature 120 can be deposited, etched, grown, or affixed near the pore 107 of the membrane 106.

The plasmonic feature 120 can be made of various materials. The plasmonic feature 120 can include one or more plasmonic materials that can enhance local electromagnetic fields. Examples of such plasmonic materials for the plasmonic feature 120 includes graphene, silicon nitride, titanium nitride, aluminum scandium nitride, or vanadium dioxide. In general, there can be one or more plasmonic feature 120 adjacent to the pore 107. The one or more plasmonic features 120 can be regularly or irregularly arranged around the pore 107.

In some implementations, the plasmonic features 120 can be deposited on the surface of the membrane 106. Example methods for depositing the plasmonic features 120 can include sputter deposition, solvent evaporation, atomic layer deposition, liquid drop casting, or chemical vapor deposition. In some embodiments, the plasmonic feature 120 can be symmetric. In other embodiments, the plasmonic feature 120 can be asymmetric. Example shapes of the plasmonic feature 120 can include roughly spherical, roughly cylindrical, roughly pyramidal, roughly toroidal, roughly oblate, or other suitable shapes.

Examples of plasmonic feature size can include from about 1 nm to about 200 nm, from about 10 nm to about 200 nm, from about 50 nm to about 200 nm, from about 100 nm to about 200 nm, from about 150 nm to about 200 nm, from about 1 nm to about 150 nm, from about 1 nm to about 100 nm, from about 1 nm to about 50 nm, or from about 1 nm to about 10 nm.

In some embodiments, the plasmonic feature 120 can be deposited adjacent to the pore 107 after pore formation. In some embodiments, one or more plasmonic features 120 can be deposited adjacent to each pore 107. For example, one or more, two or more, three or more, or four or more plasmonic features 120 can be deposited adjacent to the pore 107. In some preferred embodiments, two or more plasmonic features can be deposited adjacent to the pore 107. The two or more features can be arranged around the pore 107 in a symmetric, or an asymmetric, manner. In some embodiments, the plasmonic feature 120 can be deposited adjacent to the pore 107 prior to pore formation.

More than one electrically isolated plasmonic features spaced apart by a distance allows the distance to serve as a plasmonic “hot spot” that enhances the field, more than a singular plasmonic feature or if the area between them is conducting. The distance between the plasmonic features should generally be small to generate large field enhancement but large enough to prevent electron tunneling between features.

In some embodiments, the plasmonic feature 120 can be spaced apart from the pore 107 at a distance. Examples of the distance between the plasmonic feature 120 and the pore 107 can include a range from 1 nm to 10 nm, from 2 nm to 10 nm, from 4 nm to 10 nm, from 6 nm to 10 nm, from 8 nm to 10 nm, from 1 nm to 8 nm, from 1 nm to 6 nm, from 1 nm to 4 nm, from 1 nm to 2 nm, and other suitable distances. In some embodiments, the plasmonic feature can self-assemble at a distance from the pore 107.

The third subsystem 134 can use an optical plasmon resonance mode to detect the presence of a molecule (e.g., biomolecule (nucleic acid)). As illustrated in FIG. 1, a dark field microscopy setup can be used for to detect photons reflected, emitted, or scattered from the surface of a plasmonic feature 120 with induced surface plasmon waves. For example, the third subsystem 134 can include a light source 122 configured to emit a beam of light (e.g., collimated light) onto the surface of the plasmonic feature 120. The third subsystem 134 can include a microscope 127 that has an objective 126 configured to collect light 124 emitted, reflected, and scattered from the plasmonic feature 120. The objective 126 can include one or more lenses that can collect light.

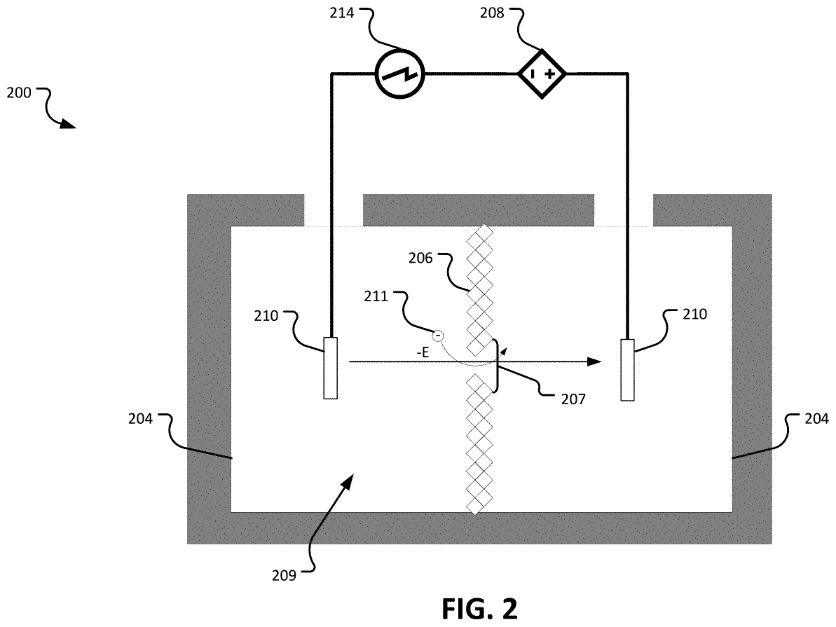

As described above, the multi-modal nanopore sequencing system 100 can detect the transit of molecules through the pore 107 of the membrane 106 using the first, second, and third subsystems 130, 132, and 134 that perform three different transit detection modes that are further described in FIGS. 2-4. In alternative implementations, the subsystem 134 can use other suitable devices (other than the microscope 127) for collecting and/or analyzing light.

The multi-modal nanopore sequencing system 100 can include a transit detection and analysis system 140. The transit detection and analysis system 140 can communicate with, and control, the components in the multi-modal nanopore sequencing system 100, such as the first, second, and third subsystems 130, 132, 134. For example, the transit detection and analysis system 140 can control the power source 108 of the first subsystem 130 to generate the electric field or current (for galvanostatic mode) across the membrane 106 between the opposite chambers 104. Further, the transit detection and analysis system 140 can receive signals from the electrical measurement device 114 (e.g., voltmeter, or ammeter) to determine the electrical signal measured in the circuit of the first subsystem 130 and analyze the signals to identify various attributes of the molecule passing through the pore of the membrane 106 as described herein.

In addition, the transit detection and analysis system 140 can control the power source 109 of the second subsystem 132 to generate the electric field or current across the pore 107 of the membrane 106. Further, the transit detection and analysis system 140 can receive signals from the electrical measurement device 115 to determine the electric field or current measured in the circuit of the second subsystem 132 and analyze the signals to identify various attributes of the molecule passing through the pore 107 of the membrane 106 as described herein. Moreover, the transit detection and analysis system 140 can control the light source of the third subsystem 134 to emit a beam 122 onto the plasmonic feature 120. Further, the transit detection and analysis system 140 can receive signals from the microscope 127 (which can contain the scattered light 124 from the plasmonic feature 120) and analyze the signals to identify various attributes of the molecules passing through the pore 107 of the membrane 106.

In some implementations, the transit detection and analysis system 140 can identify the attributes of the molecule based on the signals from the first, second, and third subsystems 130, 132, and 134, independently. In other implementations, the transit detection and analysis system 140 can identify the attributes of the molecules based on the signals from a combination of two of the first, second, and third subsystems 130, 132, and 134. In yet other implementations, the transit detection and analysis system 140 can identify the attributes of the molecules based on the signals from a combination of all of the first, second, and third subsystems 130, 132, and 134. The transit detection and analysis system 140 can further use the signal from one or more of the first, second, or third subsystems 130, 132, or 134 to verify the molecular attribute identified by a second or third subsystem.

The transit detection and analysis system 140 can be implemented by one or more computing devices. Such computing devices are intended to represent various forms of digital computers, such as laptops, desktops, workstations, personal digital assistants, servers, blade servers, mainframes, portable computer devices (e.g., wearable or mobile devices), and other appropriate computers. The computing device can include a processor, memory, a storage device, and other suitable components that run the computing device. The processor can process instructions for execution within the computing device, including instructions stored in the memory or on the storage device, so that the molecule transit detection and analysis are performed as described herein.

FIG. 2 depicts an exemplary transmembrane electrical signal system 200. The transmembrane electrical signal system 200 can perform a first detection mode of the multi-modal nanopore sequencing system 100. For example, the transmembrane electrical signal system 200 can be used for the detection of transit events through a pore 207 in a membrane 206. The transmembrane electrical signal system 200 can be used to implement the first subsystem 130 described in FIG. 1.

The transmembrane electrical signal system 200 can include a power supply 208 that can be coupled to electrodes 210 immersed in the conducting media within opposing chambers 204 of the transmembrane electrical signal system 200. The power supply 208 can apply a voltage or a current to a conducting media 209 in the chambers 204, thereby completing an electric circuit in the transmembrane electrical signal system 200.

The voltage or current applied to the electrodes 210 can generate an electric field (−E) around the pore of the membrane 206 and drive charged molecules 211 that are freely diffusing in the conducting media 209 through the pore 207 of the membrane 206. In some implementations, the transmembrane electrical signal system 200 can include an electrical measurement device 214 in the electrical circuit. The electrical measurement device 214 can measure steady electrical signal produced by the flow of ions in the conducting media 209.

As a charged molecule 211 is driven through the pore 207 of the membrane 206, the electrical signal through the electric circuit of the transmembrane electrical signal system 200 can change if the charge density of the charged molecule 211 is different than that of the surrounding conducting media 209. For example, a charged molecule 211 of different charge density than the surrounding conducting media 209 can induce a change in the steady electrical signal measured through the electric circuit. The electrical measurement device 214 in the electric circuit can detect this change as a signal.

The charge density of a charged molecule 211 can depend on size, shape, and/or charge, thereby the electrical signal in the electric circuit of the transmembrane electrical signal system 200 can change as a function of the size, shape, and/or charge of the charged molecule 211. For example, the phosphate group of a nucleotide can have a different charge density than the surrounding conducting media 209 and cause a change in the monitored electrical signal when the nucleotide is driven through the pore 207 of the membrane 206.

In some examples, the charged molecule 211 can be a nucleic acid (e.g., DNA, RNA). A nucleic acid can be composed of one or more nucleotides that form a polymer. For example, one or more, two or more, three or more, five or more, or ten or more nucleotides can form a polymer. In such embodiments, characteristics of the nucleic acid can be determined based upon the change in detected electrical signal. For example, as a first nucleotide of the nucleic acid penetrates the pore 207, a transit event is initiated and a signal can be detected. As the remaining nucleotides of the nucleic acid are driven through the pore 207 and the transit event continues, the signal will be maintained.

The signal can be maintained for the duration of the transit event (e.g., the time interval that at least one nucleotide of the nucleic acid is within the pore 207). As the final nucleotide of the nucleic acid is driven through the pore 207 and the nucleic acid is no longer within the pore 207, the transit event can be completed and the signal may return to a pre-transit level.

In some implementations, the duration of the transit event can be used to determine the number of nucleotides in a nucleic acid. In some embodiments, the change in transmembrane electrical signal (during, e.g., the duration of the transit event) can be used to determine the sequence of the nucleic acid.

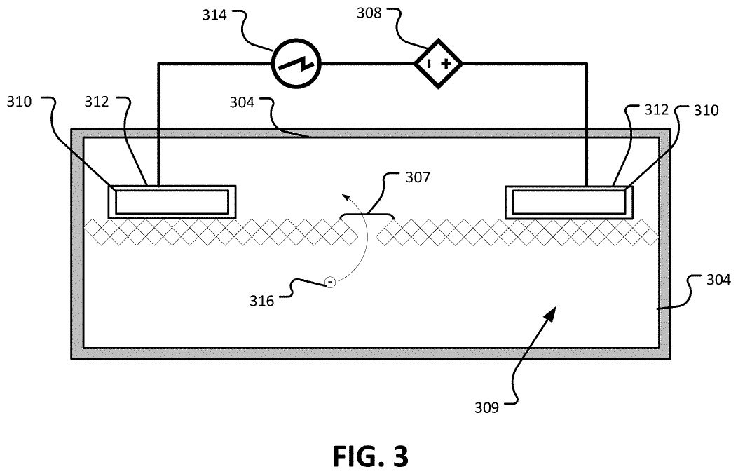

FIG. 3 depicts an exemplary trans-channel electrical signal system 300. The trans-channel electrical signal system 300 can perform a second detection mode of the multi-modal nanopore sequencing system 100. The trans-channel electrical signal system 300 can be used for the detection of transit events. For example, the trans-channel electrical signal system 300 uses the electrical signal along the planar axis of a membrane (e.g., trans-channel electrical signal) to detect the transit of a charged molecule through a pore of the membrane. The trans-channel electrical signal system 300 can be used to implement the second subsystem 132 described in FIG. 2.

The trans-channel electrical signal system 300 can include a membrane 306 within opposing chambers 304. The membrane 306 includes a pore 307 and is made of a material as described above with reference to the membrane 106, 206. The membrane 306 can separate the chambers 304 that contains a conducting media. The trans-channel electrical signal system 300 can further include a power supply 308 that can be coupled to electrodes 310. The electrodes 310 can be coated with an insulating material 312. Such coating can help preventing or reducing interference or cross-talk (e.g., leakage current) with the conducting media as well as the circuit across the membrane. The insulating material 312 can isolate the electrodes 310 from the surrounding conducting media 309 such that only the trans-channel electrical signal can be measured. The power supply 308 can apply a constant voltage or current to the membrane 306 through the electrodes 310, thereby completing an electric circuit across the membrane 306.

As illustrated in FIG. 3, the electrodes 310 of the trans-channel electrical signal system 300 electrically contact one face of the membrane 306. In general, the electrodes 310 can electrically contact one face of the membrane 306, or both of the opposite faces of the membrane 306. In some embodiments, the electrodes 310 can be on the same face, different faces, or both faces of the membrane 306. In some embodiments, the electrodes 310 can span from one face of the membrane 306 to the opposite. In some embodiments, the electrodes 310 can contact one or more edges of the membrane 306.

As described above, the electric field generated around the pore 307 of the membrane 306 can drive any charged molecules 316 that are freely diffusing in the solution 309 through the pore 307 of the membrane 306. The charged molecules 316 can be of various types as described herein. For example, the charged molecules 316 can be the molecules described in FIG. 1 or the charged molecules 211 described in FIG. 2. As a charged molecule is driven through the pore 307 of the membrane 306 of the trans-channel electrical signal system 300, the electrical signal (e.g., current, voltage, etc.) measured at the electrical measurement device 314 can change as the charged molecule 316 interacts with the electric field of the membrane 306. The change in the electrical signal can be used to determine the number or type of nucleotides in a nucleic acid, or a combination thereof. In some embodiments, the change in the electrical signal can be used to determine the sequence of the nucleic acid.

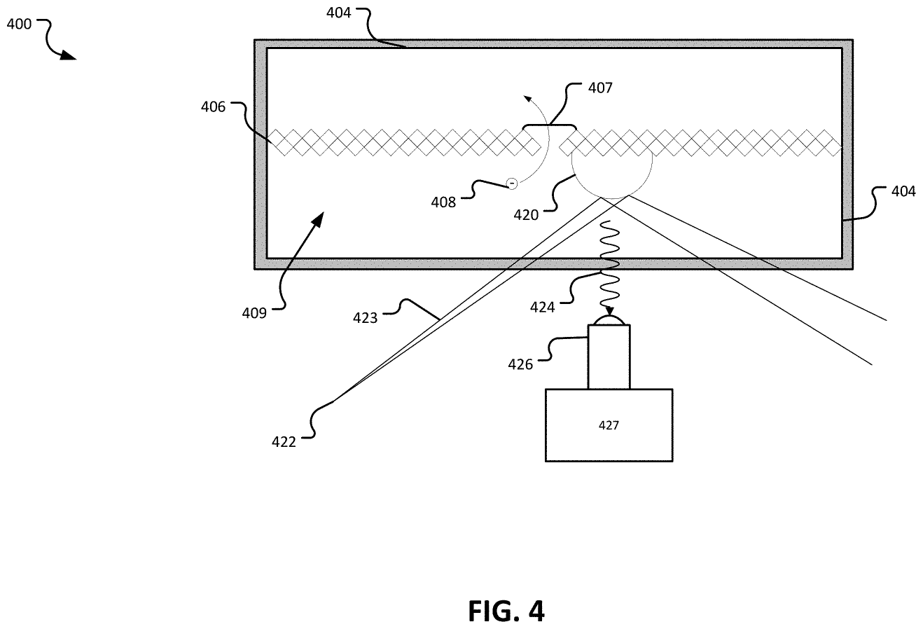

FIG. 4 illustrates an example surface plasmon detection system 400 for the detection of transit events. The surface plasmon detection system 400 can perform a third detection mode of the multi-modal nanopore sequencing system 100.

The surface plasmon detection system 400 can include a plasmonic feature 420 near a pore 407 of the membrane 406. The membrane 406 can be disposed separating two chambers 404 filled with conducting media 409. The plasmonic feature 420 can be any shape, size, or material as described herein. The plasmonic feature 420 can be positioned adjacent to the pore 407 of the membrane 406 at any distance described herein.

The surface plasmon detection system 400 can include a light source 422 that emits a beam 423 of light onto the surface of the plasmonic feature 420. Generally, the light may be collimated but it is not required. The beam 423 can have wavelength and momentum adapted to excite surface plasmon waves. For example, the light source 422 can emit light in the ultra-violet, visible, or infra-red range. Example ranges of the emitted light 423 can include between about 300 nm and about 1 mm, between about 500 nm and about 1 mm, between about 800 nm and about 1 mm, between about 1 μm and about 1 mm, between about 300 μm and about 1 mm, between about 500 μm and about 1 mm, between about 800 μm and about 1 mm, between about 300 nm and about 800 μm, between about 300 nm and about 500 μm, between about 300 nm and about 300 μm, between about 300 nm and about 1 μm, between about 300 nm and about 800 nm, or between about 300 nm and about 500 nm.

As depicted in FIG. 4, the light 423 can be substantially reflected away from the objective 426. As the beam 423 of light induces surface plasmon waves on the surface of the plasmonic feature 420, the excited surface plasmon waves can emit photons 424, scatter photons 424, or both. The photons 424 emitted by the plasmonic feature 420 in the direction of an objective 426 of a microscope 427 can be collected as a signal.

The surface plasmon detection system 400 can operate an optical detection mode to detect the presence of a molecule 408 undergoing transit through the pore 407. In some embodiments, dark field microscopy can be used to detect the presence of the molecule 408. During a transit event in which a charged molecule 408 is transiting through the pore 407 with an adjacent plasmonic feature 420, the excited surface plasmons of the plasmonic feature 420 can undergo resonant electric field interactions with the charged molecule 408 (e.g., biomolecule (nucleic acid)) undergoing the transit.

The resonant interaction can cause a change in the wavelength of photons 424 reflected, scattered, or emitted by the plasmonic feature 420 that can be collected by the objective 426. The change in wavelength of the photons 424 can be used to detect the transit of the molecule 408 (e.g., biomolecule (nucleic acid)) through the pore 407 of the membrane 406. In some embodiments, the change in frequency of the photon 424 can be used to determine the characteristics of the molecule 405 (e.g., size, shape, chemistry) during the transit event. In some embodiments, the change in frequency of the photon 424 can be used to determine the sequence of the molecule 405 (e.g., nucleic acid) during the transit event.

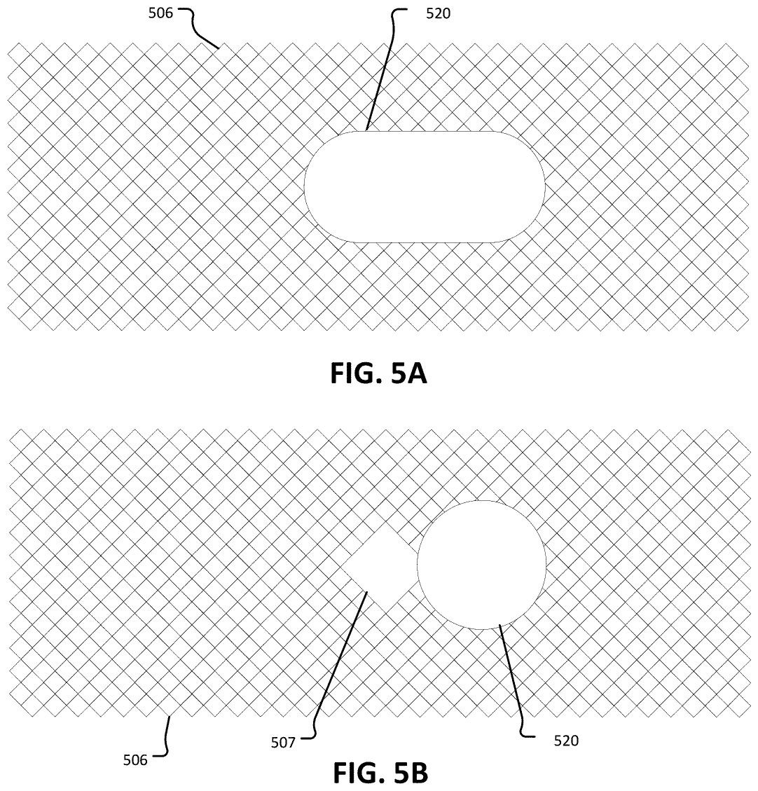

FIGS. 5A-B illustrate an example deposition of a plasmonic feature 520 on a membrane 506 in the multi-modal nanopore sequencing system 100. The deposition of the plasmonic feature 520 shown in FIGS. 5A and 5B can be used for the plasmonic features, or plasmonic feature pattern, described herein, such as plasmonic features 120, 420.

Referring to FIG. 5A, in some embodiments, the plasmonic feature 520 can be deposited on the membrane 506 prior to pore formation at the membrane 506. The plasmonic feature 520 can be any suitable shape, size, or material, similarly to those for the plasmonic features 120, 420 as described herein. After the plasmonic feature 520 is deposited on the membrane 506, in some embodiments, one or more pores 507 (FIG. 5B) can be created in the membrane 506 through heating of the deposited plasmonic feature 520.

FIG. 5A depicts the membrane 506 that has had the plasmonic feature 520 that is deposited on a surface of the membrane 506. In some embodiments, the plasmonic feature 520 can be heated to about the melting temperature of the plasmonic feature 520 material. For example, a gold plasmonic feature can be heated to a temperature in a range from about 700° C. to about 1,000° C. The heating of the plasmonic feature can cause the distortion of the shape of the plasmonic feature 520 through surface tension and radiative forces. In some embodiments, the peak temperature of the plasmonic feature 520 can be above the oxidative temperature of the membrane 506 material. For example, the melting temperature range of a gold plasmonic feature 520 (e.g., about 700° C. to about 1,000° C.) is above the oxidation temperature of a graphene membrane (e.g., about 400° C. to about 500° C.).

In some implementations, based on at least one of plasmonic feature surface tension, radiative forces, or oxidation of the molecular composition of the membrane 506, the pore 507 can be created in the surface area of the membrane 506 that the plasmonic feature 520 previously occupied. The presence of the plasmonic feature 520 adjacent to the pore 507 can be used in surface plasmon transit detection modes as described herein, for example with reference to FIGS. 1 and 4.

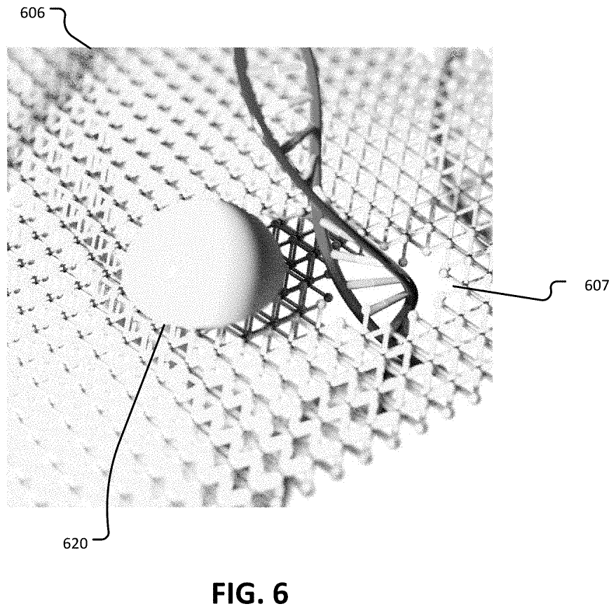

FIG. 6 depicts an exemplary membrane 606 with a single deposited plasmonic feature 620 adjacent to a pore 607. The exemplary plasmonic feature 620 is depicted as spherical and the pore 607 is undergoing a double-stranded nucleic acid transit event. The exemplary membrane 606 may be used in the multi-modal nanopore sequencing system 100 for transmembrane electrical signal, trans-channel electrical signal, or plasmon resonance detection, or any combination thereof, as described herein, for example with reference to FIGS. 1-4. The plasmonic feature 620 with the membrane 606 can be used to perform the plasmon resonance detection as described herein, for example with reference to FIGS. 1 and 4.

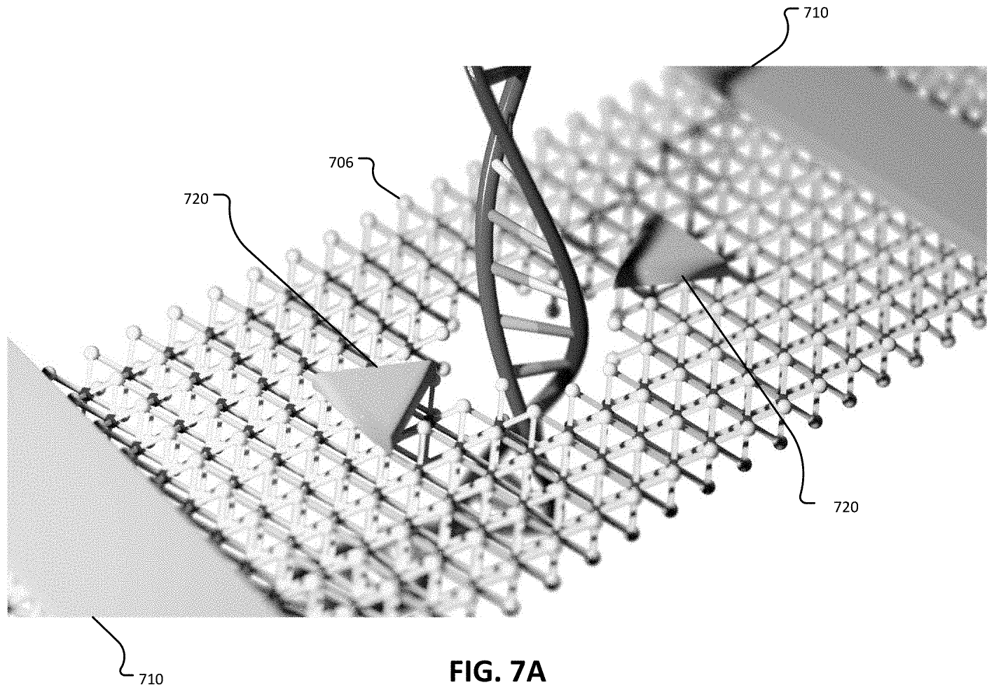

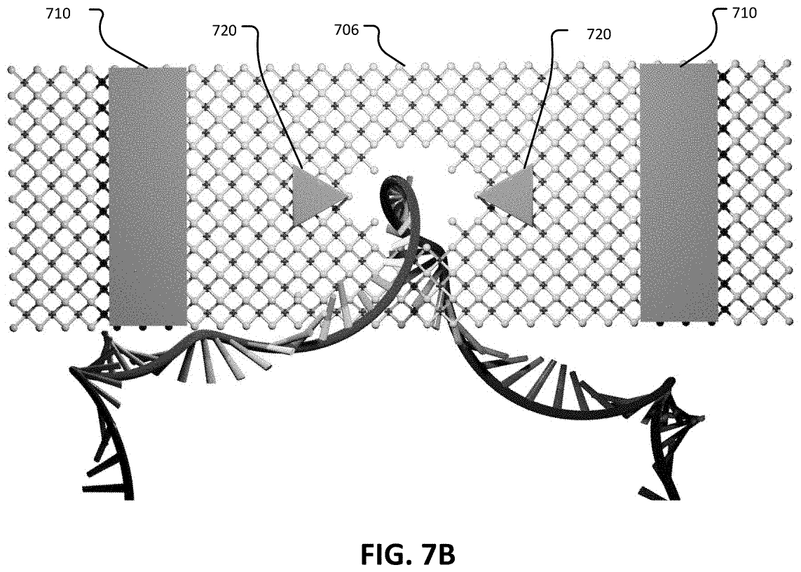

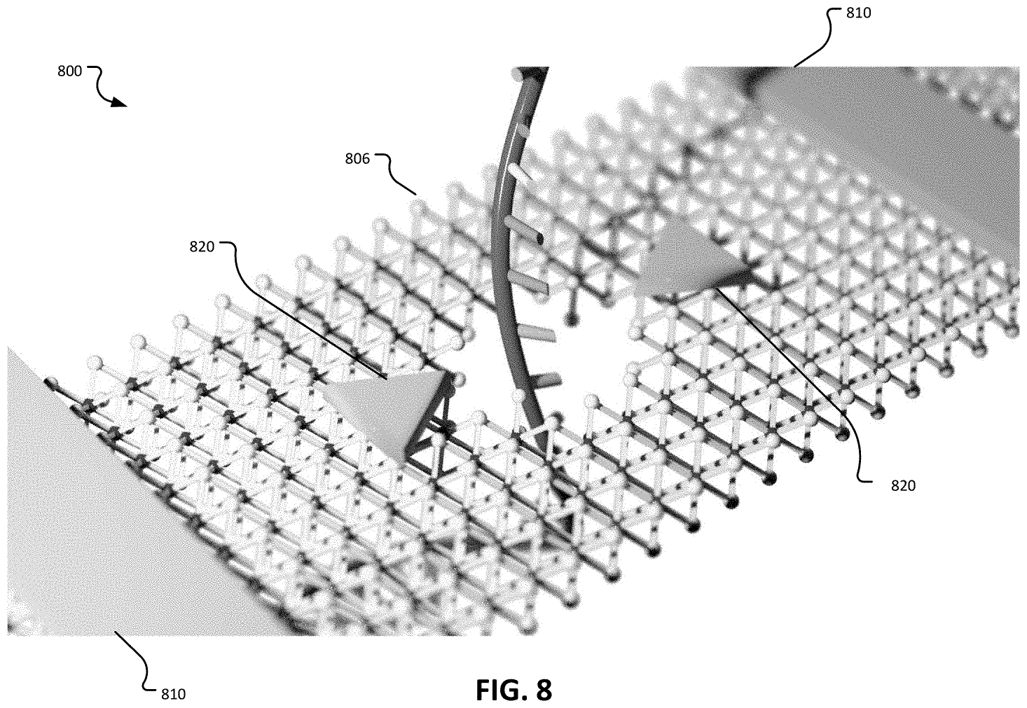

Referring to FIGS. 7A-C, an example of the multi-modal nanopore sequencing system 100 is described. In particular, FIG. 7A depicts an oblique view of an exemplary trans-channel electrical signal system 700 with two electrodes 710. FIG. 7B depicts a face view of the trans-channel electrical signal system 700 of FIG. 7A. FIG. 8 depicts an oblique view of the trans-channel electrical signal system 700 of FIG. 7A undergoing a single-stranded biopolymer transit event.

The trans-channel electrical signal system 700 can be used to implement the second subsystem 132 in FIG. 1 or the trans-channel electrical signal system 300 in FIG. 3.

In addition, the trans-channel electrical signal system 700 of FIG. 7A includes two plasmonic features 720 with a triangular profile. The two plasmonic features 720 are shown disposed adjacent to a pore 707 of an exemplary membrane 706. The trans-channel electrical signal system 700 can perform a double-stranded biopolymer transit event. The plasmonic features 720 with the membrane 706 can be used to perform the plasmon resonance detection as described herein, for example with reference to FIGS. 1 and 4. In alternative implementations, the trans-channel electrical signal system 700 includes plasmonic features that are obliquely or tangentially arranged about the pore.

The membrane 706 may be used in the multi-modal nanopore detection system 100 for transmembrane electrical signal, trans-channel electrical signal, or surface plasmon transit detection, or any combination thereof, as described herein, for example with reference to FIGS. 1-4.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. An apparatus for identifying molecules, the apparatus comprising:
a) a membrane that defines first and second chambers, the membrane including a pore that permits a molecule to pass through and move between the first and second chambers;
b) a first set of electrodes disposed in the first and second chambers respectively and configured to generate a first electric field in a conducting media between the first and second chambers;
c) a first current detector configured to measure a first current between the first set of electrodes;
d) a second set of electrodes electrically contacting the membrane and configured to generate a second electric field across the pore of the membrane;
e) a second current detector configured to measure a second current between the second set of electrodes;
f) a plasmonic feature disposed on the membrane adjacent to the pore;
g) a light source configured to emit light onto the plasmonic feature;
h) a light collector configured to collect the light scattered from the plasmonic feature; and
i) at least one computing device configured to identify at least one attribute of the molecule that passes through the pore of the membrane based on at least one of the first current, the second current, and the scattered light.

2. The apparatus of claim 1, wherein the membrane is a two-dimensional membrane.

3. The apparatus of claim 2, wherein the two-dimensional membrane is graphene or silicon-based material.

4. The apparatus of claim 1, wherein the membrane is comprised of two or more stacked membranes.

5. The apparatus of claim 1, wherein the first set of electrodes of step c) are insulated with a dielectric material.

6. The apparatus claim 1, wherein the plasmonic feature is deposited on a membrane lacking pores.

7. The apparatus of claim 6, wherein the plasmonic feature is used to create the pore.

8. The apparatus of claim 1, wherein a diameter of the plasmonic feature is between 1 nm and 100 nm.

9. The apparatus of claim 1, wherein the plasmonic feature is a metallic plasmonic feature.

10. The apparatus of claim 6, wherein the plasmonic feature is deposited by chemical vapor deposition.

11. The apparatus of claim 1, wherein the molecule that passes through the pore of the membrane is a nucleic acid.

12. The apparatus of claim 11, wherein the at least one attribute of the molecule is a sequence of the nucleic acid.

13. The apparatus of claim 1, wherein the attribute of the molecule includes at least one of a size, a charge, or a weight of the molecule.

14. The apparatus of claim 1, wherein the at least one computing device is configured to identify the at least one attribute of the molecule based on surface plasmon resonance.

15. A method for detecting a molecule attribute, the method comprising:
a) providing an apparatus comprising:
a. a membrane including a pore that permits a molecule to pass;
b. a first set of electrodes disposed at opposite sides of the membrane and configured to generate a first electric field in a conducting media in a direction through the pore of the membrane;
c. a second set of electrodes electrically contacting the membrane and configured to generate a second electric field across the pore of the membrane;
d. a plasmonic feature disposed on the membrane adjacent to the pore;
e. a light source configured to emit light onto the plasmonic feature;
b) measuring at least one of:
a. a first signal representative of a first electric current or voltage supplying between the first set of electrodes;
b. a second signal representative of a second electric current or voltage supplying between the second set of electrodes; or
c. a third signal representative of a light scattered from the plasmonic feature deposited near the pore; and
c) determining a molecule attribute based on the at least one of the first signal, the second signal, or the third signal.

16. The method of claim 15, wherein the molecule attribute is a size of the molecule.

17. The method of claim 16, wherein the molecule is a nucleic acid comprising DNA or RNA.

18. The method of claim 17, wherein the molecule attribute is a sequence of the nucleic acid.

19. The method of claim 15, wherein measuring the light scattered from the plasmonic feature comprises using microscopy.

20. The method of claim 15, wherein determining the molecule attribute comprises:
determining the molecule attribute based on one or two of the first signal, the second signal, and the third signal; and
verifying the molecule attribute based on the remaining of the first signal, the second signal, and the third signal.
